(19) United States
(12) Patent Application Publication
(10) Pub. No.: US 2007/0179812 A1
(43) Pub. Date: Aug. 2, 2007

(54) Health history formatting method and system for the same

(76) Inventor: Joseph Chapman, Washington, DC (US)

Correspondence Address: THE HARRIS FIRM, 2830 SOUTH MEADE, ARLINGTON, VA, 22206, US

(21) Appl. No.: 11/340,600

(22) Filed: Jan. 27, 2006

(51) Int. Cl. G06F 19/00; A61B 5/00

(52) U.S. Cl. 705/3; 600/300

(57) ABSTRACT

A health history formatting system, comprising at least one computer server connected to a remote user interface via the Internet, wherein the health history formatting system: (a) elicits health history data from a patient, who personally transmits the data to the health history formatting system; (b) stores the data for subsequent maintenance by the patient; (c) formats the data for review by a healthcare professional; and (d) provides the patient with a printable version of the formatted data; all without ever requesting or storing any of the patient's personally identifiable or identifying information. A method for implementing the same is also disclosed.

### FIELD OF THE INVENTION

The present invention relates to a health history formatting method, and a system for achieving such method. More particularly, the present invention relates to a health history formatting method, and a related system, which provide efficient and safe, creation and dissemination of a patient's health history.

### BACKGROUND OF THE INVENTION

Healthcare professionals need comprehensive, accurate, rapidly-reviewable and easily-assimilated information concerning a patient's health. To this end, healthcare professionals and their assistants traditionally gather and record health information by interviewing patients or their surrogates (parents, guardians, caregivers or other authorized persons) and by systematizing data according to health topics in headings and subheadings. The health topics are usually described in detailed narratives to provide essential characteristics such as the duration, severity, and quality of a topic. Healthcare professionals subsequently review the narrative and reconstruct the history to understand a patient's health status better, to perceive the development of any unhealthy condition, and to anticipate any future health problems. This method has proven to be effective but very time consuming. A method that decreases the time required for a healthcare professional to gather, assimilate and record the health history of patients could thus improve safety, increase productivity, and reduce the cost related to healthcare.

Several obstacles stand in the way of achieving these goals, however. Healthcare providers sometimes have patients complete a questionnaire that may identify some of a patient's health information. Nevertheless, some of these questions are not phrased with great detail, may be incompletely answered, or may not be answered at all, which requires additional time to obtain and record this unanswered information during the patient's visit.

As patients encounter new healthcare professionals, moreover, the information must again be recalled by the patient and communicated through the same process. At best, the patient's medical records are forwarded from one healthcare professional to another, but the patient must still provide current information for completion of such records. In some cases, much vital information is lost or distorted in the process, especially with each successive recall.

Perhaps most importantly, the risk of compromising the privacy and security of sensitive patient health information must be minimized. Without the promise of such security, patients often withhold disclosure of certain vitally important facts out of fear that such information might, for instance, endanger their employment, insurability, or even credit rating. Thus, it is important that patients have control over the accessibility and comprehensiveness of their respective health histories.

Several attempts electronically to format health history data exist. United States patent application number 2003/0088439, for example, discloses a magnetic floppy disk that contains a database management software program and related, patient-specific data, which is entered and maintained by the patient. However, this invention is a burden to carry, is susceptible to being damaged by the patient, and is difficult to update as new medical advances cause health professionals to seek out different types of information. It also fails to allow rapid data assimilation by healthcare professionals.

Hence, the prior art fails to provide an electronic health history system or method that is patient-controlled, and that enables a patient or his surrogate, to create a comprehensive health history not-susceptible to easy destruction or loss by a patient.

### SUMMARY OF THE INVENTION

Thus, the present invention is directed to a health history formatting system that provides complete control over a patient's health history to the patient, without requiring the patient to carry or store the information, and thereby risk the destruction or loss of the information by the patient.

The present invention is likewise directed to a health history formatting method that provides complete control over a patient's health history to the patient, without requiring the patient to carry or store the information, and thereby risk the destruction or loss of the information by the patient.

One aspect of the present invention is directed to a health history formatting system comprising at least one computer server connected to a remote user interface via the Internet, wherein the health history formatting system: elicits health history data from a patient, who sends the data to the health history formatting system; stores the data for subsequent maintenance by the patient; formats the data for review by a healthcare professional; and provides the patient with an electronic or printable version of the formatted data; all without ever requesting or storing any of the patient's personally identifiable information.

In another aspect, the health history formatting system issues the patient a randomly-generated patient identification number that is unique to the patient.

In yet another aspect, the health history formatting system relates the data to the patient identification number.

In still another aspect, the health history formatting system formats the data chronologically in columns for rapid review and assimilation by a healthcare professional or others authorized by the patient.

In yet another aspect, the health history formatting system issues the patient only a randomly-generated patient password that is unique to the patient.

In still another aspect, the health history formatting system relates the data to the patient password.

In yet another aspect, the health history formatting system allows the patient to download formatted health history and send it to a healthcare professional electronically.

In still another aspect, the health history formatting system provides the patient up-to-date and comprehensive lists of health related options, the particulars of which might otherwise be overlooked or forgotten by the patient.

In yet another aspect, the health history formatting system provides the patient with an option to exclude certain sensitive or extraneous data from a printed report or electronically sent report.

Another aspect of the present invention is directed to a health history formatting system comprising at least one computer server connected to a remote user interface via the Internet, wherein the medical history formatting system: enables review of health related topics by a patient or surrogate, who records relevant topics with associated date and description data in the health history formatting system; stores the data for subsequent review, editing and maintenance by the patient or surrogate; formats the data chronologically in columns and compartments for rapid review and assimilation by a healthcare professional or others authorized by the patient; and provides a version of the formatted data that can be printed directly or downloaded to a protocol that allows off-line word-processing, storage and transmittal; all without ever requesting or storing any of the patient's personally identifiable or identifying information.

Another aspect of the present invention is directed to a health history formatting method comprising the steps of: eliciting health history data from a patient, who personally transmits the data to the health history formatting system; storing the data for subsequent maintenance by the patient; formatting the data for review by a healthcare professional; and providing the patient with a printable version of the formatted data; all without ever requesting or storing any of the patient's personally identifying information.

### BRIEF DESCRIPTION OF THE DRAWINGS

In the accompanying drawings, which form a part of the specification and are to be read in conjunction therewith, and in which like reference numerals are used to indicate like parts in the various views:

FIG. 1 is a schematic diagram of one embodiment of the health history formatting system of the present invention, and several data transmission pathways and user interfaces with which it may be used; and

FIG. 2 is a schematic diagram of some of the steps performed by the system of FIG. 1; and

FIG. 3 is an illustration of a health history report produced using the health history formatting system of FIG. 1.

### DETAILED DESCRIPTION OF THE INVENTION

As illustrated in the accompanying drawings and discussed in detail below, one aspect of the present invention is directed to a health history formatting system that enables patients anonymously to create, store, edit, print, download and maintain an organized health history from a Web site at no cost to the patient or healthcare professional. In particular, this aspect allows the patient to create an accurate and comprehensive health history through tutorials, instruction, and systematized lists of health topics—without limiting such instruction or options to the confines of an easily-damaged, destroyed, or lost patient-held device. The potential scope of such health history is therefore, unlimited both in its size and comprehensiveness, and it is easily-updated on a remote server. This aspect, moreover, provides safe and secure remote storage of the patient's health history that can be accessed at any time with only a unique identification number and/or secure, user password.

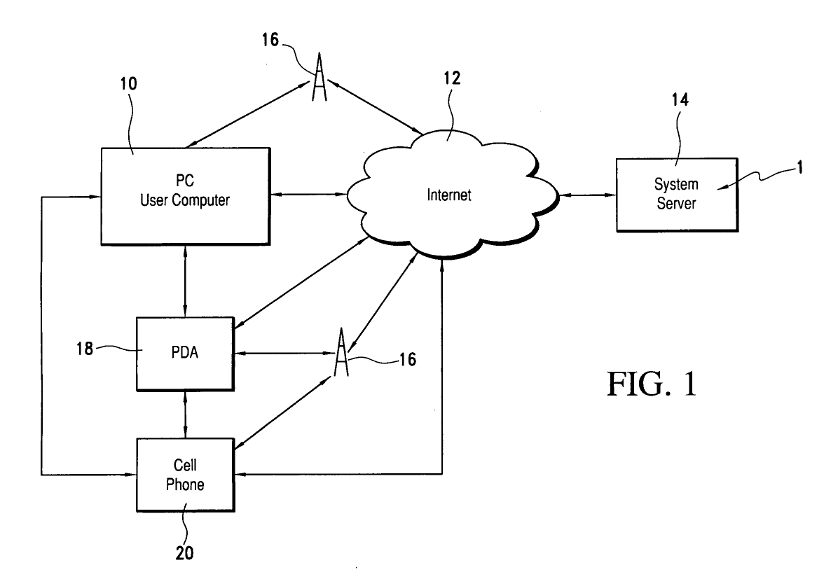

In one embodiment, this aspect provides application service provider (“ASP”) software on a remote server that can be accessed through multiple pathways. Referring to FIG. 1, system server 14, on which ASP software is stored, performs the functions of health history formatting system 1. Systems suitable for use with this embodiment also include multiple server environments and other data processing and storage devices, however.

Users, who comprise individual patients, parents, guardians, or other authorized persons, access and interact with system 1 through the Internet 12 from a variety of devices and pathways. Suitable interface devices include the user's personal computer 10, PDA 18 or cell phone 20, all of which may connect with each other or the Internet 12. Other devices suitable for this embodiment include any electronic device that can receive, send, and display data so that it can be understood by a user.

Thus, communication pathways suitable for use in this embodiment include, but are not limited to, cell towers 16, the Internet 12, and any electronic, magnetic, or optical transmission medium suitable for transmitting health history data between a user and system 1.

In one embodiment, the software code that provides functionality to system 1 is created by using Apache® freeware and Java®Script. Many other software design programs are suitable for use in this embodiment, however.

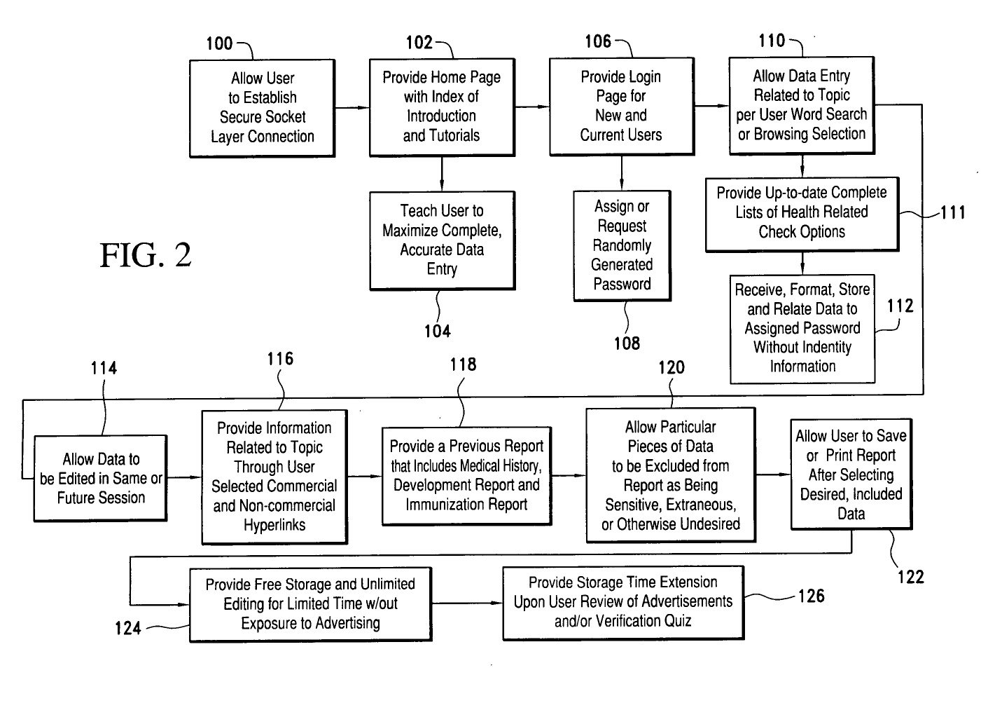

Referring to FIG. 2, system 1 allows 100 the user to establish a secure socket layer (“SSL”) connection between the user's PC 10 interface device and system 1. System 1 provides 102 a home page with an index of introduction and tutorials that teach 104 the user various information such as the purpose and general approach to using system 1's Web site, and graphic, video, audio and text demonstrations of the various elements of the Web site. In one embodiment, during these tutorials the user is taught the importance of comprehensive and accurate health records, as well as various medical distinctions and clarifications to obtain the same.

System 1 provides 106 a login page for new and current users. In the case of a new user, system 1 assigns 108 a randomly-generated identification number that is unique to the patient. It also requires that the user create a login password to access patient data. Upon subsequent visits to the Web site, system 1 prompts 108 a current user to enter previously generated, assigned, and issued identification number, along with the user created password, to login.

In one embodiment, the identification number is sequentially-generated. However, many other number or symbol generating methods are suitable for use in this aspect of the invention.

In one embodiment system 1 assigns 108 only a randomly-generated or sequentially-generated alphanumeric password to the data. Such password is also used for user login. In this embodiment a separate user-generated password is not required, therefore, because the password functions both to link patient data to a patient, and to provide a patient secure access to such data. Any number or symbol suitable for anonymously linking patient data to a particular patient may be used in this aspect of the invention.

System 1 allows 110 the user to enter data related to a health topic according to a user word search (e.g., by typing-in a topic), or according to user browsing through a selection of health topics and various subordinate descriptors.

System 1 enhances patient safety by providing 111 up-to-date, comprehensive lists of health-related options that are selected or left unselected by the user, such as by selecting a “check” box with a PC cursor. One of these lists includes allergies and sensitivities that might otherwise be forgotten. System 1 prompts the user to select from a full and accurate list of symptoms, signs and diagnosis that might conflict with a proposed treatment for a new illness. It further presents a comprehensive list of current or past medical treatments and devices in use, or used, that could conflict with a proposed treatment of a new illness including, but not limited to, medications, therapies, surgeries, and prostheses. Another list provides an opportunity to enter family health problems that might affect the patient.

At no time does system 1 request or accept data that would identify the patient or user, such as, but not limited to, name, address, phone number, driver's license number, or Social Security number.

Therefore, because it comprehensively elicits patient information, system 1 reduces the possibility for unnecessary diagnostic tests, repeat visit(s) or delayed diagnosis and treatment.

System 1 receives, formats, and stores patient data and assigns the data to the assigned identification number without requesting or saving any personally identifiable or identifying information. System 1 stores the data so that it can be presented in a concise and medically-meaningful format to a healthcare professional. In particular, system 1 stores patient data so that it can be chronologically-listed with relevant dates, health topics, and descriptions related to each respective health topic, presented in separate columns and compartmentalized by horizontal and vertical lines into boxes. Thus, once a report is generated and presented to a healthcare professional as described below, system 1 further provides cost savings by preparing health history documents in a format that can be readily assimilated.

System 1 allows 114 the user to edit patient data in the same (i.e., current) session, or during a future login session.

System 1 provides 116 information related to a particular health topic through user selected commercial or non-commercial hyperlinks to outside Web sites, without the presence of intrusive or annoying banners or pop-up ads.

System 1 provides 118 the user with a preview report that includes the patient's medical history report, development report, immunization report, and other reports based on queries that provide useful subsets of information from the patient's database. System 1 formats patient data at the user interface device so that it is chronologically-listed with relevant dates, health topics, and descriptions related to each respective health topic, presented in separate columns and compartmentalized by horizontal and vertical lines into boxes. This non-narrative format is arranged irrespective of organ systems.

System 1 allows 120 particular pieces of data to be excluded from the report by the user. Such data may be excluded for example if it is sensitive, extraneous, or otherwise not wanted in the report for any other reason.

System 1 allows 122 the user to save, electronically send, and/or print the report once it is previewed and after the user selects the data that is to be included in the report.

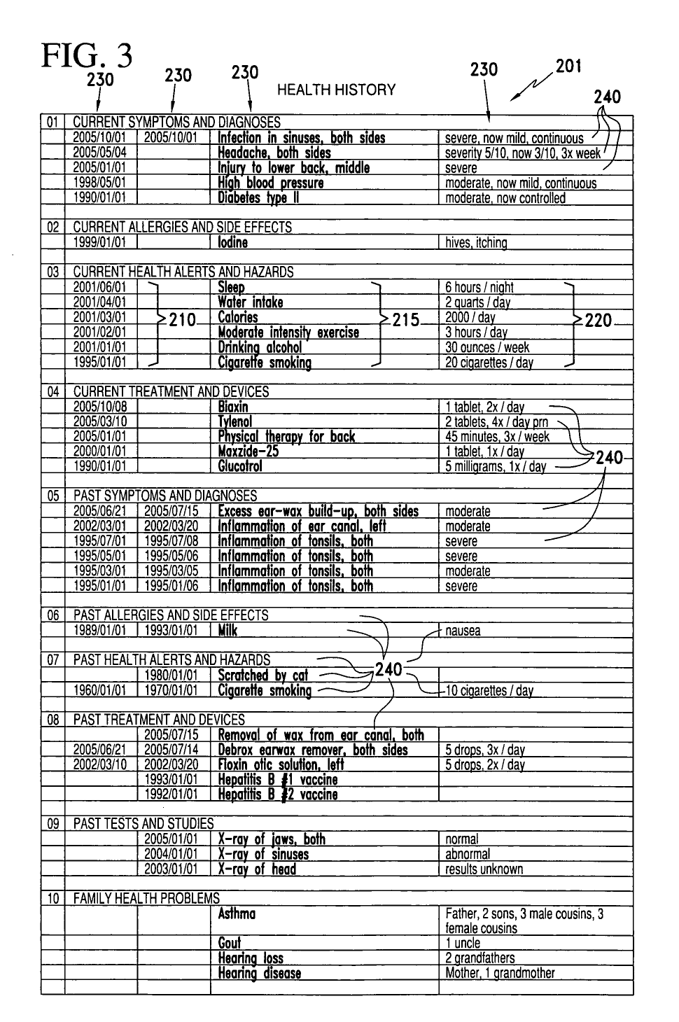

Referring to FIG. 3, system 1 provides formatted paper report 201, which, when presented to a healthcare provider, enables the healthcare provider to obtain more information in a shorter period of time than by personally interviewing a patient. System 1 thus provides a user with patient data in a paper print out that is chronologically-listed with relevant dates 210, health topics 215, and descriptions 220 related to each respective health topic, presented in separate columns 230 and compartmentalized by horizontal and vertical lines into boxes 240. This non-narrative format is arranged irrespective of organ systems. As such, diagnosis and treatment can begin more quickly once report 201 is reviewed by a healthcare professional.

In one embodiment, system 1 allows a user electronically to send a health history report to a healthcare provider as an e-mail message attachment. Such report is formatted like paper report 201; it is chronologically-listed with relevant dates 210, health topics 215, and descriptions 220 related to each respective health topic, presented in separate columns 230 and compartmentalized by horizontal and vertical lines into boxes 240. This non-narrative format is arranged irrespective of organ systems. As such, diagnosis and treatment can begin more quickly once the electronic report or paper report 201 is reviewed by a healthcare professional.

System 1 provides 126 free storage and unlimited login and editing for a limited time, e.g., 7 days, without requiring the user to contribute to the support of the Web site.

In one embodiment, system 1 further provides 126 an additional storage time extension for users who contribute to the support of the Web site, by allowing them to review, through hyperlinks, the Web sites of vendors who pay for the opportunity to market their products and services. Users confirm their participation by correctly answering several questions in a quiz derived from information provided on a vendor's Web site. Users have no obligation to purchase products or services and are requested not to submit any personally identifying or identifiable information to the vendors Web site while logged-in to this Web site. System 1 accordingly provides users the facility to create a comprehensive and organized health history without cost or risk of loosing anonymity.

A second aspect of the present invention is directed to a health history formatting method, several of the embodiments of which are substantially described above.

While it is apparent that the illustrative embodiments of the invention disclosed herein fulfill the objectives of the present invention, it is appreciated that numerous modifications and other embodiments may be devised by those skilled in the art. Additionally, feature(s) and/or element(s) from any embodiment may be used singly or in combination with other embodiment(s). Therefore, it will be understood that the appended claims are intended to cover all such modifications and embodiments that would come within the spirit and scope of the present invention.

I claim:

1. A health history formatting system, comprising:
at least one computer server connected to a remote user interface via the Internet;
wherein the health history formatting system:
(A) elicits health history data from a patient, who sends the data to the health history formatting system;
(B) stores the data for subsequent maintenance by the patient;
(C) formats the data for review by a healthcare professional; and
(D) provides the patient with an electronic or printable version of the formatted data;
all without ever requesting or storing any of the patient's personally identifiable information.

2. The system of claim 1 wherein the health history formatting system issues the patient a randomly-generated patient identification number that is unique to the patient.

3. The system of claim 2 wherein the health history formatting system relates the data to the patient identification number.

4. The system of claim 3 wherein the health history formatting system formats the data chronologically in columns for rapid review and assimilation by a healthcare professional or others authorized by the patient.

5. The system of claim 1 wherein the health history formatting system issues the patient only a randomly-generated patient password that is unique to the patient.

6. The system of claim 5 wherein the health history formatting system relates the data to the patient password.

7. The system of claim 1 wherein the health history formatting system allows the patient to download formatted health history and send it to a healthcare professional electronically.

8. The system of claim 1 wherein the health history formatting system provides the patient up-to-date and comprehensive lists of health related options, the particulars of which might otherwise be overlooked or forgotten by the patient.

9. The system of claim 1 wherein the health history formatting system provides the patient with an option to exclude certain sensitive or extraneous data from a printed report or electronically sent report.

10. A health history formatting system, comprising:
at least one computer server connected to a remote user interface via the Internet;
wherein the medical history formatting system:
(A) enables review of health related topics by a patient or surrogate, who records relevant topics with associated date and description data in the health history formatting system;
(B) stores the data for subsequent review, editing and maintenance by the patient or surrogate;
(C) formats the data chronologically in columns and compartments for rapid review and assimilation by a healthcare professional or others authorized by the patient; and
(D) provides a version of the formatted data that can be printed directly or downloaded to a protocol that allows off-line word-processing, storage and transmittal;
all without ever requesting or storing any of the patient's personally identifiable or identifying information.

11. A health history formatting method, comprising the steps of:
eliciting health history data from a patient, who personally transmits the data to the health history formatting system;
storing the data for subsequent maintenance by the patient;
formatting the data for review by a healthcare professional; and
providing the patient with a printable version of the formatted data;
all without ever requesting or storing any of the patient's personally identifying information.
